(19) United States
(12) Patent Application Publication
(10) Pub. No.: US 2004/0162274 A1
(43) Pub. Date: Aug. 19, 2004

(54) Inhibition of steroid synthesis by use of progesterone or a progesterone receptor agonist

(76) Inventors: Hans-Joachim Paust, Hamburg (DE); Amal K. Mukhopadhyay, Hamburg (DE); Vladimir Patchev, Jena (DE)

Correspondence Address: ARNOLD & PORTER LLP, ATTN: IP DOCKETING DEPT., 555 TWELFTH STREET, N.W., WASHINGTON, DC, 20004-1206, US

(21) Appl. No.: 10/697,565

(22) Filed: Oct. 31, 2003

(30) Foreign Application Priority Data
Nov. 1, 2002 (DE) 10251028.8

(51) Int. Cl. A61K 31/57

(52) U.S. Cl. 514/177

(57) ABSTRACT

The present invention relates to the use of progesterone or a progesterone receptor agonist for the inhibition of steroid synthesis, in particular for inhibiting the expression of the steroidogenic acute regulatory protein (StAR protein).

### CROSS-REFERENCE TO RELATED APPLICATIONS

This application claims benefit under 35 U.S.C. §119 of German Patent Application No. 10251028.8, filed Nov. 1, 2002, the disclosure of which is hereby incorporated by reference in its entirety.

### BACKGROUND OF THE INVENTION

The present invention relates to the use of progesterone or a progesterone receptor agonist for the inhibition of steroid synthesis, in particular for inhibiting the expression of steroidogenic acute regulatory protein (hereinafter referred to as “StAR”).

Progesterone or 4-pregnene-3,20-dione is the most important natural gestagen and is formed primarily in the corpus luteum and in the placenta. In males, this hormone is formed in the adrenal cortex and in the testicles.

Progesterone is a steroidal hormone and acts together with the oestrogens on the reproductive organs and it is involved in nearly all female reproductive functions. This hormone therefore determines the luteal phase of the menstrual cycle, prepares the organism for a pregnancy, creates important prerequisites for conception and nidation and has a thermogenetic effect independent of oestrogens. The formation of the steroid hormones is essential for human survival and the reproductive function. The biosynthesis of the steroid hormones can take place under basal conditions or by means of acute or chronic control using trophic hormones, which are formed in the pituitary gland (Menon et al., Fertil. Steril., 25:732-750 (1974); Waterman et al., Vit. Horm., 52:129-1548 (1996)). Binding of trophic hormones to the seven-transmembrane-domain G-protein coupled receptor (7 TM GPCR) increases the activity of adenylate cyclase, which leads to the formation of cAMP. cAMP promotes steroid formation by inducing steroidogenic genes (Menon et al., Fertil. Steril. 25:732-750 (1974), Waterman et al., Vit. Horm., 52:129-1548 (1996)).

Steroids are synthesised from specialised steroidogenic cells in the adrenal gland, the ovaries, the placenta, the testicles and the brain. Although steroidal hormones have different physiological effects, the biosynthesis of all steroids starts with the conversion of cholesterol to pregnenolone. This reaction is catalysed by the cytochrome P450 side-chain cleavage enzyme (hereinafter referred to as “P450_{scc}”), which is localised on the matrix side of the inner mitochondrial membrane (Farkash et al., Endocrinology, 118; 1353-1365 (1986)). For many years, it was assumed that the activity of the P450_{scc} was the rate-limiting step in steroid production. If there is an insufficient quantity of the substrate cholesterol, the activity of this enzyme can indeed be the rate-limiting factor. However, it was later discovered that the activity of the enzyme is not the actual regulating step but rather the transport of the substrate, cholesterol, to the inner mitochondrial membrane and to the P450_{scc} (Brownie et al., Biochem. Biophys. Res. Commun., 46:483-490 (1972), Simpson et al., J. Biol. Chem. 253:3135-3139 (1979), Crivello et al., J. Biol. Chem., 255:8144-8151 (1980), Privalle et al., Proc. Natl. Acad. Sci. USA, 80:702-706 (1983)).

A basic observation of these investigations consisted in the synthesis of new proteins as an absolute prerequisite for the regulation of steroidal synthesis (Ferguson, Biochem. Biophys. Acta., 57:616-617 (1962), Ferguson, J. Biol. Chem., 238:2754-2759 (1963)). This conclusion was based on the observation that protein synthesis inhibitors, including those resulting from trophic hormones such as the adrenocorticotrope hormone (ACTH) and the luteinizing hormone (LH), inhibited induced steroid formation. Further studies showed that the inhibition of protein synthesis had no effect on the transport of cellular cholesterol to the outer mitochondrial membrane. The transport of the substrate from the outer membrane to the inner membrane was, however, completely inhibited (Privalle et al., Proc. Natl. Acad. Sci. USA, 80:702-706 (1983), Ohno et al., Endocrinology, 30:355-228 (1983)).

Consequently, a regulatory protein was postulated, which would affect the transport of the cholesterol from the outer mitochondrial membrane via the aqueous, inner membrane area to the inner membrane. Due to the hydrophobic nature of cholesterol, a transfer to P450_{scc} would not be possible in the quantities necessary for the observed amount of steroidal synthesis after hormonal stimulation. Such transport could not take place without a transport facilitator. Evidence suggests that the StAR protein (steroidogenic acute regulatory protein) is the transport facilitator and regulates the steroid formation.

StAR was originally described by Orme-Johnson and colleagues (Brownie et al., Biochem. Biophys. Res. Commun., 46:483-490 (1972)). It was identified as a rapidly inducible 30 kDa phosphoprotein in ACTH-treated rats and adrenal cells of the mouse as well as in LH-treated cells of the corpus luteum of the rat and Leydig cells of the mouse. Later, it was described in hormone stimulated MA-10 mouse Leydig tumour cells by Stocco and colleagues (Clark et al., J. Biol. Chem., 269:8314-28322 (1993), Stocco et al., Endocr. Rev., 17:221-244 (1996), Stocco et al., Steroids, 62:29-36 (1997)).

The StAR protein was localized in mitochondria and consists of different forms of a 30 kDa protein newly synthesised under the influence of gonadotropin. In addition to the 30 kDa protein, precursor forms of this protein with 37 kDa and different N-terminal signal sequences were discovered (Epstein and Orme-Johnson, J. Biol. Chem., 266:19739-19745 (1991), Stocco et al., J. Biol. Chem., 266:19731-19738 (1991)).

The cDNA coding for the 37 kDa mitochondrial protein was cloned and expressed by means of transient transfection in MA-10 and COS-1 cells, which after transfection were capable to produce steroids. This process led to a multiple increase of the conversion from cholesterol to pregnenolone (Stocco et al., Endocr. Rev., 17:221-244 (1996); Clark et al., J. Biol. Chem. 269:8314-28322 (1994), Sugawara et al., Proc. Natl. Acad. Sci. USA, 92:4778-4782 (1995), Lin et al., Science, 267:1828-1831 (1995)). These results show an immediate cause-effect relationship between the 37 or 30 kDa proteins and the hormone-regulated steroid synthesis.

Moreover, an essential role for StAR in steroid formation is suggested by the observation that mutations in the StAR gene result in the possibly fatal condition known as congenital lipoid adrenal gland hyperplasia (lipoid CAH) (Lin et al., Science, 267:1828-1831 (1995)). Patients with this condition are not able to produce sufficient amounts of steroids. Such patients have an excessive amount of cholesterol and cholesterol ester in the adrenal gland and in testicular steroid-forming cells. Such Patients can only survive by means of a suitable steroid replacement therapy.

StAR knockout mice have been produced that have a phenotype essentially corresponding to that of lipoid CAH (Caron et al., Proc. Natl. Acad. Sci. USA, 94:11540-11545 (1997)). Biochemical and genetic investigations therefore consistently show that StAR has an important role in steroid hormone biosynthesis in tissues of the adrenal gland and of the gonads. Testicular Leydig cells are dependent on a chronic stimulation by means of LH for obtaining their structure and the steroid-forming function (Saez, Endocr. Rev., 15:574-626 (1994)). The capability of the Leydig cells to form steroids is reduced during ageing (Luo et al., J. Andrology, 17:509-515 (1996)). The ageing process includes the formation of reactive oxygen radicals, which damage lipids, proteins and/or DNA (Stadtman, Science, 257:1220-1224 (1992)). Damage by reactive oxygen species (ROS) also leads to a functional senescence. It has been shown that this ROS damages important constituents of the steroid biosynthesis path (Quinn et al., J. Biol. Chem., 259:4130-4135 (1984), Quinn et al., J. Biol. Chem., 260:2092-2099 (1985)). Further, it has been observed that ageing Leydig cells exhibit an increased production of progesterone and a reduced formation of testosterone.

There are a large number of conditions, in which adrenal steroid synthesis is abnormally increased due to pathologically activated stimulation mechanisms. A natural and reliable control of steroid biosynthesis would be desirable for the treatment of these disease conditions and also for other medicinal purposes.

The problem underlying the present invention, thus, resides in providing means for the inhibition of steroid biosynthesis in as natural a way as possible and which means are therefore especially suited for the treatment of pathological conditions.

This problem is solved according to the invention by the use of progesterone or a progesterone receptor agonist to inhibit the expression of the StAR gene.

### BRIEF SUMMARY OF THE INVENTION

The present invention includes and provides a method of reducing StAR gene expression comprising administering an amount of progesterone receptor agonist to a patient in need thereof, wherein said StAR gene expression is reduced.

A preferred embodiment of the present invention includes a method of reducing StAR gene expression comprising administering an amount of progesterone receptor agonist to a patient in need thereof, wherein said StAR gene expression is reduced, where a progesterone receptor agonist includes, for example without limitation, progesterone, R5020, and drospirenone.

The present invention includes and provides a sample from a patient with a pathological condition of steroid synthesis, wherein said patient is treated with a progesterone receptor agonist, and wherein StAR gene expression is reduced in said patient.

The present invention includes and provides a sample from a patient with a pathological condition of steroid synthesis, wherein said sample is treated with a progesterone receptor agonist, and wherein StAR gene expression is reduced in said sample.

A preferred embodiment of the present invention includes and provides use of progesterone or a progesterone receptor agonist for inhibiting the expression of the StAR gene. A more preferred embodiment of the present invention includes use of progesterone or a progesterone receptor agonist for inhibiting the expression of the StAR gene, where inhibition of the expression of the StAR gene is used for the treatment of mammals, in particular for therapeutic or prophylactic treatment.

A most preferred embodiment of the present invention includes and provides use of progesterone or a progesterone receptor agonist for inhibiting the expression of the StAR gene, where inhibition of the expression of the StAR gene by means of progesterone or a progesterone receptor agonist is used for the treatment of a pathological condition of steroid synthesis.

A most preferred embodiment of the present invention includes use of progesterone or a progesterone receptor agonist for inhibiting the expression of the StAR gene according to any of the preceding embodiments, where the progesterone receptor agonist is R5020 or drospirenone.

A preferred embodiment of the present invention includes use of progesterone or a progesterone receptor agonist for inhibiting the expression of the StAR gene according to any of the preceding embodiments, where inhibiting the expression of the StAR gene involves a reduction in the expression of the StAR gene by at least 20%, preferably by at least 30% and particularly preferably by at least 50%.

A preferred embodiment of the present invention includes use of progesterone or a progesterone receptor agonist for inhibiting the expression of the StAR gene according to any of the preceding embodiments for the treatment of a condition in which adrenal steroid synthesis is abnormal due to pathologically activated stimulation.

A preferred embodiment of the present invention includes use of progesterone or a progesterone receptor agonist for inhibiting the expression of the StAR gene according to any of the preceding embodiments for the treatment of a condition, which involves a pathologically increased activity of the adrenal cortex, and which results from a congenital or medically induced glucocorticoid receptor resistance.

A particularly preferred embodiment of the present invention includes and provides use of progesterone or a progesterone receptor agonist for inhibiting the expression of the StAR gene according to any of the preceding embodiments, where a pathological condition includes, for example and without limitation, chronic stress, alcohol deprivation, endogenous depression, ACTH and/or gonadotropin-secreting tumours of the adenohypophyseal or its metastases, ectopic ACTH syndrome (bronchial carcinoids), prostate hyperplasia, cancer, micronodular adrenal disease, congenital adrenal hyperplasia (CAH), pubertas praecox, virilising syndrome in women and polycystic ovary with proven androgen hypersecretion.

A particularly preferred embodiment of the present invention includes use of progesterone or a progesterone receptor agonist for inhibiting the expression of the StAR gene according to any of the preceding embodiments, where the treatment is used for mammals. A more particularly preferred embodiment of the present invention includes use of progesterone or a progesterone receptor agonist for inhibiting the expression of the StAR gene according to any of the preceding embodiments, where the treatment is used for humans.

A particularly preferred embodiment of the present invention includes use of progesterone or a progesterone receptor agonist for inhibiting the expression of the StAR gene according to any of the preceding embodiments, where the progesterone or progesterone receptor agonist is administered orally. The progesterone or progesterone receptor agonist can be administered orally in a variety of ways known in the art. For example without limitation, the progesterone or progesterone receptor agonist can be administered orally in the form of a tablet, or parenterally, for example by subcutaneous, intravenous or intraperitoneal injection or catheterisation.

### BRIEF DESCRIPTION OF THE DRAWINGS

FIG. 1 shows the effect of DHT, estradiol and progesterone on cAMP production in isolated Leydig cells of rats.

FIG. 2 shows the effect of R5020 and progesterone on the hCG-induced cAMP production in isolated Leydig cells of rats.

FIG. 3 shows the effect of R5020 and drospirenone on the hCG-induced testosterone and cAMP production in isolated Leydig cells of rats.

FIG. 4 shows the effect of progesterone on the expression of StAR protein in Leydig cells in vitro.

### DEFINITIONS

As used herein, the term “progesterone receptor agonist” designates a compound, which suggests a measurable, specific binding to the progesterone receptor and, after successful binding of the receptor, contributes a significant change to the transcription of defined gestagen-responsive genes. For example and without limitation, a progesterone receptor agonist includes gestagens such as R5020 and drospirenone.

As used herein, the term “progesterone (INN)” refers to the compound 4-pregnene-3,20-dione (IUPAC).

As used herein, the term “condition” refers to a pathological condition of steroid synthesis when the basal steroid synthesis over an extended period of time, preferably over a period of 24 hours, is increased by at least 20% or by at least about 20%, preferably by at least 30% or by at least about 30%, and particularly preferably by at least 50% or by at least about 50% in relation to the amount of steroid synthesis of a healthy individual of the same age.

### DETAILED DESCRIPTION OF THE INVENTION

It was surprisingly established that a steroid hormone, namely progesterone or a progesterone receptor agonist, is especially suitable for inhibiting the expression of the StAR gene. Progesterone and progesterone receptor agonists therefore provide natural, reliable, and highly effective inhibitors of StAR expression, which leads to an inhibition of steroid synthesis, which is independent of the inhibition of supra- adrenal factors (such as corticotropins or gonadotropins).

In agreement with the standard meaning, the term progesterone (INN) is used in the present application to refer to the compound 4-pregnene-3,20-dione (IUPAC).

Further, the term “progesterone receptor agonist” in the context of the present invention designates a compound, which provides a measurable, specific binding to the progesterone receptor and, after successful binding of the receptor, contributes a significant change to the transcription of defined gestagen-responsive genes. Examples of a progesterone receptor agonist are gestagens such as R5020 and drospirenone.

Inhibition of StAR gene expression is observed when StAR gene expression in a sample from a treated subject is reduced by at least 20% and by at least about 20% relative to a corresponding sample from the same subject before treatment or in comparison with a corresponding sample from an untreated subject. For example without limitation, the sample can be a sample of tissue.

The expression of the StAR gene is reduced by at least 20% or by at least about 20%, preferably by at least 30% or by at least about 30%, and particularly preferably by at least 50% or by at least about 50%. The scope of the invention naturally also includes the complete inhibition of the expression of the StAR gene, i.e. an inhibition that reduces the quantity of StAR transcripts below a detectable level in a sample from a subject.

The inhibition of gene expression can be established by the quantitative determination of the StAR mRNA or by determining the amount of StAR protein in tissues or samples. A large number of methods for the quantitative determination of mRNA, and for the quantitative determination of proteins are known to the person of ordinary skill and can be used in the context of the present invention to determine the inhibition of StAR gene expression. For example without limitation, northern blot, RT-PCR, nucleic acid chips, etc., can be used to quantitatively determine mRNA, and western blot, protein chips, etc., can be used to quantitatively determine proteins.

The present invention involves the use of progesterone or a progesterone receptor agonist for inhibiting the expression of the StAR gene for the treatment of mammals, in particular for the therapeutic or prophylactic treatment of humans.

According to a particularly preferred embodiment of the present invention, inhibition of the StAR gene expression by means of progesterone or a progesterone receptor agonist is used for the treatment of a pathological condition of steroid synthesis. In the context of the present invention, a condition is described as a pathological condition of steroid synthesis when the basal steroid synthesis over an extended period of time, preferably over a period of 24 hours, is increased by at least 20% or by at least about 20%, preferably by at least 30% or by at least about 30%, and particularly preferably by at least 50% or by at least about 50% in relation to the amount of steroid synthesis of a healthy individual of the same age. The amounts of individual steroids normally expected in a healthy individual are sufficiently well known to a person skilled in the art when particular parameters, for example, such as age and sex of an individual, are taken into about. Moreover, standard normal values or ranges of the amounts of steroids are published in the textbook “Practical Endocrinology” by Allolio and Schulte, “Praktische Endokrinologie”, Urban & Fischer Verlag, Appendix XV, 730-736 (1996).

The use of progesterone or a progesterone receptor agonist according to the present invention can, therefore, be used particularly for the treatment of a pathological condition, where the pathological condition includes abnormally increased adrenal steroid synthesis due to a pathological stimulation, or for the treatment of a condition, which involves a pathologically increased activity of the adrenal cortex, which results from a congenital or medically induced glucocorticoid receptor resistance (cf., for example, Kamilaris and Chrousos, “Adrenal diseases”, In: Diagnostic Endocrinology, B. C. Decker Inc., 79-109 (1990)). The pathological condition can be, for example without limitation, chronic stress, alcohol deprivation, endogenous depression, ACTH and/or gonadotropin-secreting tumours of the adenohypophyseal or its metastases, ectopic ACTH syndrome (bronchial carcinoids), prostate hyperplasia, cancer, micronodular adrenal disease, congenital adrenal hyperplasia (CAH), pubertas praecox, virilising syndrome in women or poly cystic ovary with proven androgen hypersecretion.

In the context of the present invention, the progesterone or progesterone receptor agonist can be administered to a subject by any known method in the state of the art. The amount to be administered depends on the objective of the treatment and the active ingredient and can be determined by the person skilled in the art using standard methods. The amount of progesterone or progesterone receptor agonist to be administered may, for example without limitation, range from 2-5 mg/day for certain medical uses.

A large number of administration forms may be used, among which oral administration, for example in the form of a tablet, and parenteral techniques such as subcutaneous, intravenous and intraperitoneal injections, catheterisations and the like are preferred. Furthermore, administration can also be achieved via a skin plaster or by nasal or other forms of administration through the mucous membrane.

The present invention relates to methods for the manufacture of a medicinal product, which contains progesterone or a progesterone receptor agonist and which is to be used for inhibiting the expression of the StAR gene. All substances required for the manufacture of the medical product can be obtained commercially (e.g. drospirenone from Schering AG, Germany; R5020, obtainable as promegestone, from NEN Life Sciences, Boston, Mass.).

The dosing regimen and method of administration of the progesterone or progesterone receptor agonist can be determined by the average person skilled in the art with regard to the planned medicinal indication by methods known from the state of the art.

According to a further embodiment of the present invention, the use of progesterone or a progesterone receptor agonist for the manufacture of a medical product for the therapeutic or prophylactic treatment of a condition is provided, the condition is characterised by an increased amount of steroids in the patient.

### EXAMPLES

Chemicals and reagents were procured from the following manufacturers: Human chorionic gonadotropin (hCG) was procured from Boehringer Mannheim (Mannheim, Germany). Steroids, such as dihydrotestosterone (DHT), estradiol and progesterone were obtained from Sigma (Taufkirchen, Germany). R5020 NEN Life Sciences and drospirenone were procured from Jenapharm (Jena, Germany). Albumin fraction V (from bovine serum) was obtained from Merck (Darmstadt, Germany). All other reagents were procured from commercial suppliers and complied with the highest degree of purity.

### Example 1

### Isolation and Purification of Leydig Cells

The testicles of young (three months old) and old (23-24 months old) Wistar rats (Charles River, Sulzfeld, Germany) were removed immediately after killing the rats by means of carbon dioxide anaesthesia and cervical dislocation.

In order to isolate and to purify the Leydig cells from the testicles of young and old rats, the decapsulated testicular mass of healthy Wistar rats was treated with collagenase (0.25 mg/ml) for 30 minutes at room temperature as described by Mukhopadhyay et al., Biochem. J., 239:463-467 (1986a), Mukhopadhyay et al., FEBS Lett., 202:111-116 (1986b)). The homogenous cell suspension so obtained was filtered through a nylon filter to obtain an impure Leydig cell preparation. After further purification by means of a Percoll gradient, the final number of high-grade purified Leydig cells was determined using a Neubauer chamber.

Aliquots of 100,000 cells per incubation tube were used for the experiments in which cAMP was measured. The cells were pre-incubated for 3 hours and subsequently incubated for 30 minutes with or without the addition of different concentrations of hCG as a surrogate for LH. In order to investigate the effect of the different steroids, Leydig cells were subjected to steroids for 3 hours as described. The cells were then stimulated with hCG (3 ng/ml) for 30 minutes. Incubation was stopped by the addition of absolute ethanol (final alcohol concentration ca. 80%) and subsequent mixing by means of vortices. The ethanol extract was evaporated until dry, and the residue dissolved in 1 ml of MEM, which contained 0.1% sodium azide. The amount of cAMP formed was determined by means of a specific ELISA, which was carried out as described in Budnik and Mukhopadhyay, FEBS Letters, 419:4-8 (1997). However, commercially available ELISAs, such as, for example, CM 59221 (IBL, Hamburg, Germany) or DE 0355 or DE 0450 (R&D Systems GmbH, Wiesbaden, Germany) can also be used.

Similar culture conditions as described above were used for experiments, in which testosterone was measured. However, aliquots of 50,000 cells per incubation tube were used. The cells were pre-incubated for 3 hours with different steroids or without steroids and subsequently incubated for 30 minutes in the absence or in the presence of hCG. Incubation was stopped by the addition of absolute ethanol (final alcohol concentration ca. 80%) and subsequent mixing by means of vortices. The ethanolic extract was evaporated until dry, and the residue was dissolved in 1 ml of MEM, which contained 0.1% sodium azide. The content of testosterone was determined by means of specific, commercially available kits (IBL, Hamburg). The amount of testosterone for 100,000 cells was calculated on the basis of the measured values.

### Example 2

### Extraction of RNA and Northern Hybridization

RNA was isolated from 1.5×106 high-quality cleaned Leydig cells, which were cultivated in 12-well plates (750,000 cells/well). The RNA was extracted in accordance with a modified single-step method of Chomczynski and Sacchi, Anal. Biochem., 162:156-159 (1987), using the small-scale phenol-free RNA isolation kit (Ambion Inc., Austin, Tex.). According to the size, the isolated RNA was separated by gel electrophoresis in a 1.2% agarose gel using the formaldhyde/morpholino-propane sulphonic acid method (Sambrook et al., Vol.1, 7.37, 7.39-7.52, Nolan, C. (Ed), Cold Spring Harbor Laboratory Press, Cold Spring Harbor, N.Y.). The transfer (blotting) of the RNA from the gel onto a nylon membrane (nytran, Schleicher and Schüll, Dassel, Germany) was carried out by capillary transfer using a downwards-directed transfer for 4 hours in accordance with the manufacturer's instructions (Ambion Inc., Austin, Tex.; AMS Biotechnology, Wiesbaden, Germany) with modifications by Chomczynski, P., Anal. Biochem., 201:134-139 (1992).

A specific hybridizing probe for the nucleic acid sequence encoding the StAR protein was amplified by means of PCR using a 5′ primer and a 3′ primer, which, in the published cDNA sequence (Clark et al., J. Biol. Chem. 269:8314-28322 (1994)), are located in positions corresponding to nucleotides 339-360 and in positions corresponding to nucleotides 581-600. The nucleic acid sequence encoding the StAR protein was sub-cloned into the cloning vector, pCR™II (Invitrogen, Groningen, The Netherlands). In order to provide evidence of transcripts in Leydig cells, which are used to obtain the nucleic acid sequence encoding the StAR protein, antisense RNA was transcribed in vitro from a cDNA fragment (1 μg), which was amplified by the isolated plasmid DNA that contained a 262 base pair insert of the StAR protein gene under the control of a T7 promoter. An M13 forward primer and the specific 5′ primer of the StAR protein were used. The transcription reaction was carried out under conditions, which were in accordance with the Ambion protocol, using [α-¹² P]UTP (ca. 800 Ci/mmol) (obtainable from Amersham-Pharmacia Biotech, Freiburg, Germany). For hybridization, 1×106 cpm/ml were used for 18 hours at 68° C. followed by washing steps with lower and high stringency at 68° C. in 2×SSC, 0.1% SDS and 0.1×SSC, 0.1% SDS, respectively. The blots were subjected to an autoradiographic film or phosphoimaging detection using a Storm 860 apparatus (Molecular Dynamics, Amersham Biosciences, Freiburg, Germany).

### Example 3

### Specific Reduction of cAMP Production in Isolated Leydig Cells of Rats as a Function of Progesterone Concentration or of Progesterone Receptor Agonist Concentration

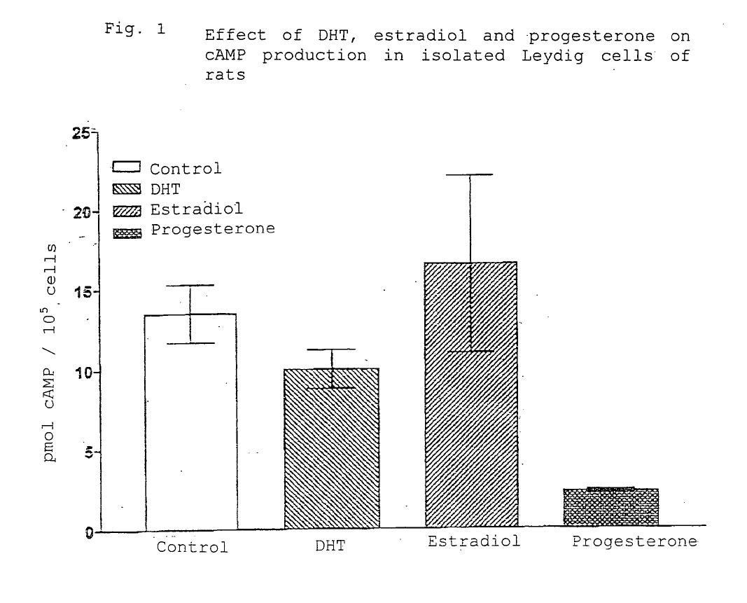

Leydig cells were subjected to 3 different steroids for 3 hours (see FIG. 1). Subsequently, the cells were stimulated over 30 minutes with hCG (human chorion gonadotropin; 3 ng/ml) as a surrogate for LH.

The amount of cAMP formed was determined using a specific ELISA.

The presence of progesterone causes the Leydig cells to significantly inhibit the cAMP reaction to gonadotropin stimulation (see FIG. 1).

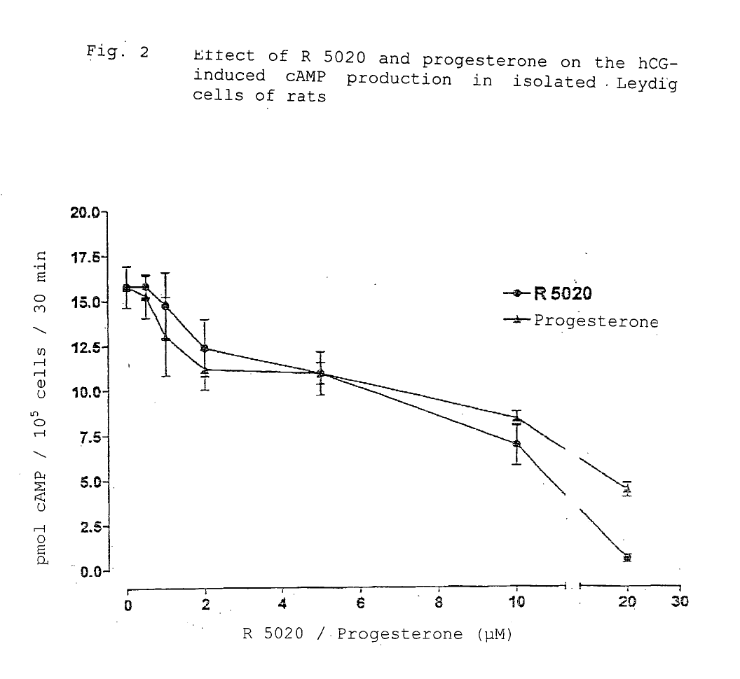

A dose-response curve was produced for the action of progesterone on the Leydig cells with regard to cAMP formation (see FIG. 2). The data represented in FIG. 2 clearly shows a dose-dependent inhibitory effect of progesterone on hCG-induced cAMP formation in Leydig cells.

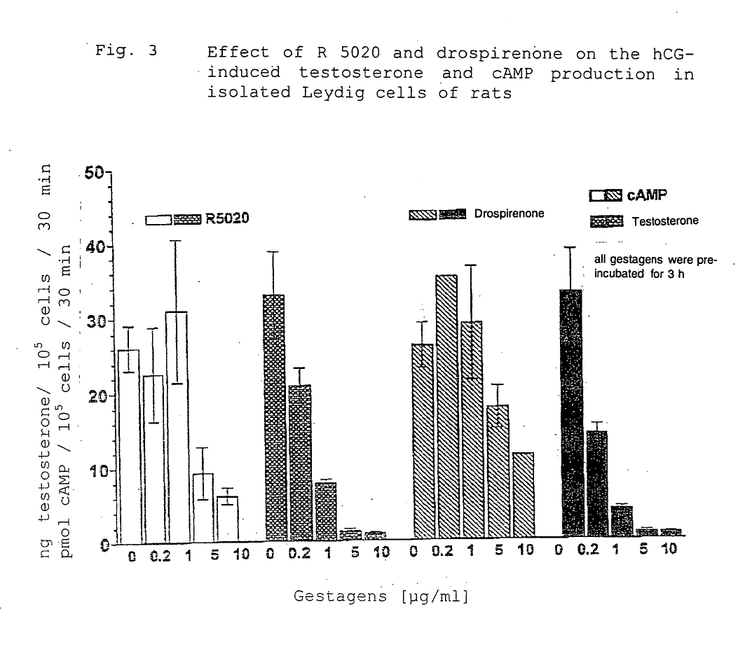

Although progesterone at a concentration of 0.1 μmol/l (10⁻⁷ mol/l) had almost no effects, higher concentrations, up to 2.5 μmol/l progesterone, showed clearly inhibitory effects. This action of the progesterone was also initiated by other gestagens such as R5020 (see FIGS. 2 and 3) and drospirenone (see FIG. 3). The dose-response curve corresponded to that of the progesterone dose-response curve (see FIG. 2). R5020 and drospirenone drastically reduced the amount of cAMP formed in the cells in response to gonadotropic stimulation.

Both R5020 and drospirenone are clearly, structurally different from testosterone. It was to be determined whether these two compounds also inhibit the hCG-induced testosterone synthesis in Leydig cells. FIG. 3 shows a test that was carried out under similar conditions to those described above for FIG. 2. In FIG. 3, the cells were treated with different concentrations of R5020 and drospirenone for 3 hours followed by stimulation with hCG (5 ng/ml) for 3 hours. Then, the quantity of testosterone that accumulated in the medium was measured. Both compounds were also able to inhibit the production of testosterone.

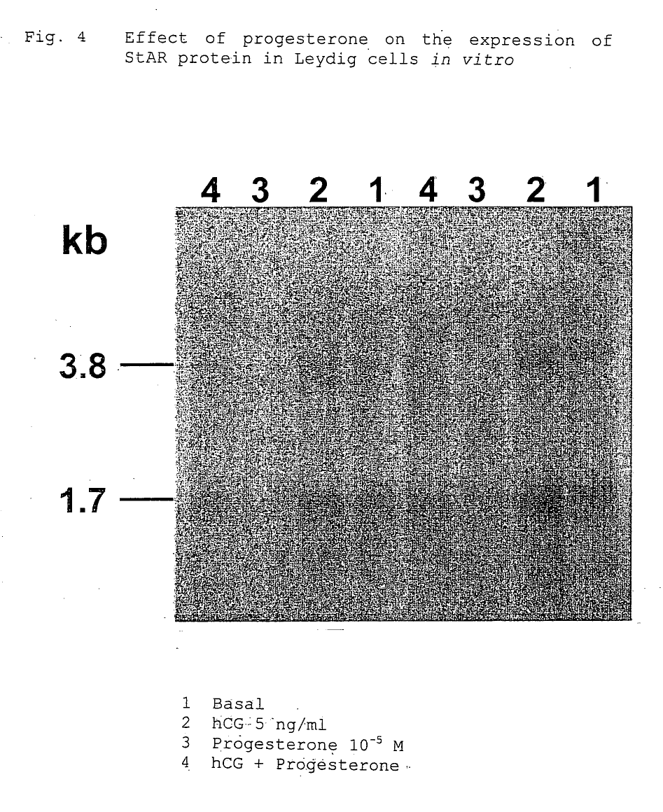

As the gestagens mentioned above apparently both have inhibitory effects on cAMP and on the production of steroids in gonadotropin-stimulated Leydig cells, the inhibitory effects of progesterone on the quantity of StAR protein expressed were analysed. In order to investigate the effect of progesterone on StAR protein expression, Leydig cells were pre-incubated for 3 hours with or without progesterone. The cells were subsequently further incubated with hCG (5 ng/ml) for 3 hours. At the end of the incubation, total RNA was extracted and StAR protein expression was analysed using northern blots. Results of two representative tests, each carried out with independent cell samples, are shown in FIG. 4.

Lane 1 shows cells, which were not treated; Lane 2 shows cells, which were treated with hCG alone. Two transcripts of the StAR protein, one with a length of 3.8 kb and another with a length of 1.7 kb, were found. Stimulation with hCG increased the expression of the transcripts.

Pre-treatment with progesterone did not significantly affect the expression of the StAR protein in those cells that were not treated with a gonadotropin (Lane 3). These cells were used in the context of the present experiment as a negative control. Progesterone does not affect the constitutive expression of the StAR protein. An inhibitory effect can be demonstrated under conditions of over-stimulation of the steroid synthesis, i.e. when cells are stimulated with supra-adrenal factors (corticotropins or gonadotropins) StAR gene expression is inhibited.

A reduction of the signal in Lane 3 could, however, be detected in comparison with Lane 1. But, as shown in Lane 4, pre-treatment with progesterone led to a significant reduction in the hCG-stimulated amounts of StAR protein (cf. Lane 4 and Lane 2). The tests described here, therefore, clearly show that treatment with progesterone causes an inhibition of the hCG-stimulated expression of StAR protein in primary Leydig cells. It has therefore been possible to show for the first time on the molecular level that progesterone inhibits the expression of the transcripts coding for the StAR protein in Leydig cells.

Having generally described the invention, the same can be more readily understood through reference to the above examples that are provided by way of illustration, and are not intended to be limiting of the present invention, unless specified. Each periodical, patent, and other document or reference cited herein is hereby incorporated by reference in its entirety.

What is claimed:

1. A method of reducing steroidogenic acute regulatory (StAR) gene expression comprising administering an amount of progesterone receptor agonist to a patient in need thereof, wherein said StAR gene expression is reduced.

2. The method of claim 1, wherein said progesterone receptor agonist is selected from the group consisting of progesterone, R5020, and drospirenone.

3. The method of claim 1, wherein adrenal steroid synthesis is abnormally increased in said patient in need thereof due to pathologically activated stimulation mechanisms.

4. The method of claim 1, wherein said patient in need thereof has a pathological condition of steroid synthesis.

5. The method of claim 1, wherein said pathological condition of steroid synthesis is selected from the group consisting of: chronic stress, alcohol deprivation, endogenous depression, ACTH-secreting tumours of the adenohypophyseal, or metastases thereof, gonadotropin-secreting tumours of the adenohypophyseal, or metastases thereof, ectopic ACTH syndrome (bronchial carcinoids), prostate hyperplasia, cancer, micronodular adrenal disease, congenital adrenal hyperplasia (CAH), pubertas praecox, virilising syndrome, and polycystic ovary with proven androgen hypersecretion.

6. The method according to claim 1, wherein said reduced StAR gene expression provides treatment to said patient in need thereof selected from the group consisting of therapeutic and prophylactic.

7. The method according to claim 1, wherein said progesterone receptor agonist is administered orally.

8. The method according to claim 1, wherein said reduced StAR gene expression is independent of inhibition of supra-adrenal factors.

9. The method according to claim 1, wherein said supra-adrenal factors are selected from a group consisting of corticotropins and gonadotropins.

10. The method according to claim 1, wherein said StAR gene expression is reduced by at least 20%.

11. The method according to claim 10, wherein said StAR gene expression is reduced by at least 30%.

12. The method according to claim 11, wherein said STAR gene expression is reduced by at least 50%.

13. The method according to claim 1, wherein said patient in need thereof has a pathologically increased activity of the adrenal cortex.

14. The method according to claim 13, wherein said pathologically increased activity of the adrenal cortex is due to a congenital glucocorticoid receptor resistance.

15. The method according to claim 13, wherein said pathologically increased activity of the adrenal cortex is due to a medically induced glucocorticoid receptor resistance.

16. A sample from a patient in need thereof with a pathological condition of steroid synthesis, wherein said patient in need thereof is treated with a progesterone receptor agonist, and wherein StAR gene expression is reduced in said patient in need thereof.

17. The sample according to claim 16, wherein said patient in need thereof is a mammal.

18. The sample according to claim 17, wherein said mammal is a human.

19. The sample according to claim 16, wherein said progesterone receptor agonist is selected from the group consisting of progesterone, R5020, and drospirenone.

20. A sample from a patient in need thereof with a pathological condition of steroid synthesis, wherein said sample is treated with a progesterone receptor agonist, and wherein StAR gene expression is reduced in said sample.
